(19) United States
(12) Patent Application Publication
(10) Pub. No.: US 2009/0030285 A1
(43) Pub. Date: Jan. 29, 2009

(54) MONITORING OF USE STATUS AND AUTOMATIC POWER MANAGEMENT IN MEDICAL DEVICES

(76) Inventor: Bjorn K. ANDERSEN, Struer (DK)

Correspondence Address: STITES & HARBISON PLLC, 1199 NORTH FAIRFAX STREET, SUITE 900, ALEXANDRIA, VA, 22314, US

(21) Appl. No.: 11/782,837

(22) Filed: Jul. 25, 2007

(51) Int. Cl. A61B 5/00; A61B 7/04; A61M 15/00; A61M 5/158

(52) U.S. Cl. 600/300; 128/200.14; 381/67; 604/272

(57) ABSTRACT

A method is provided for automatically determining the use status of an electronic medical device (e.g., an electronic stethoscope) and/or activating such a device. The method includes providing a patient portion of the device, i.e., one or more portions of the device that, in use, are brought into contact with a patient, with a contact or proximity detector. The detector provides an output signal when the patient portion is proximate to, or in contact with, a portion of a patient. This output signal, after signal processing, is used in determining the use status of the device.

### TECHNICAL FIELD

The present invention relates generally to devices and methods for automatically determining the use status of medical devices and more particularly to automatic power management based on said use status in electronic medical devices. Specifically the invention relates to power management of electronic stethoscopes, when such devices are turned on prior to the use of the stethoscope and to problems related to turning on such devices or relating to the time required for such electronic devices, such as stethoscopes, to become operable after turning on the device prior to use. Furthermore, the use status determination according to the invention may find use in other devices than stethoscopes, such as injector devices for administering medicaments or inhaling devices.

### BACKGROUND OF THE INVENTION

The use of traditional mechanical/acoustical stethoscopes is well established and such devices are by their nature immediately operable whenever desired without the need to turning on the device, i.e. to provide it with energy. More recently electronic stethoscopes have become available and such devices offer many advantages over traditional passive mechanical/acoustical devices (i.e. devices not provided with active amplification means or other signal-processing means enabling the device to carry out an active signal processing, for instance filtering of signals or analysis/evaluation of the signals picked up by these devices).

However, users of electronic medical devices such as stethoscopes often find it problematic that the device has to be turned on prior to use and some users also experience the period it takes for such devices to become operative after turning on the device as a problem. With electronic stethoscopes (or other medical devices) of the digital type, the latter problem is caused by the boot time of the digital device, i.e. the time it takes for loading software into the RAM of the device from an external Flash/E2prom memory. The associated waiting time is often experienced as inconvenient by users, although this waiting time is typically only a few seconds.

A solution to the above problems would be to maintain the device in a stand-by mode of operation in order to enable very rapid power-up of the device, but this solution is typically associated with excessive and unacceptable power consumption and hence critically shortened battery lifetime.

Furthermore, all types of new advanced electronic circuitry, for instance wireless communication, that are integrated into such devices will increase power consumption and therefore further enhance problems with a shortened battery lifetime.

Power drain may be controlled to some extent by limiting the time of operation for each activation such that the power is for Instance turned off three minutes after last button operation, assuming that this operation indicates that the device is no longer in active use, but three minutes, or other pre-selected time intervals, may possibly be much longer time than actually required, thus an examination of a patient may for instance last for only 10 to 15 seconds.

### SUMMARY OF THE INVENTION

On the above background it is an object of the present invention to provide a device or technology that is able to automatically determine the use status of a medical electronic device, such as an electronic stethoscope.

It is a further object of the invention to provide means and methods for keeping power consumption of the device low and hence increase battery lifetime.

On the above background it is a specific object of the present invention to provide means for automatically turning on an electronic medical device, such as an electronic stethoscope when the appropriate portion of the device is to be brought into an operable state. An example would be automatic turning on of the device for instance when the sound sensor of a stethoscope is nearing the skin of a patient.

The portion of the device that is brought into contact with the patient during use will collectively be referred to as the “device-operator/patient portions” throughout the present specification. This expression is used in the present context because the use status of the device and the need to activate the device can be indicated either by a given portion of the device (such as the acoustic sensor or “chestpiece” of a stethoscope) being brought in contact with a portion of a patient's body or being brought to a position at close proximity to the patient's body or by the operator of the device actually touching a portion of the device (such as the operator picking up the device or a portion hereof prior to applying it on a patient). In most of the examples of this specification, however, use status/requirement of activation of the device will be determined by a portion of the device either actually being brought into contact with a surface portion of a patient or to a position in close proximity to the patient.

According to the present invention, the above object is attained by the provision of an intelligent/automatic monitor means that monitors the use status (for instance in active use or in a stand-by or idle mode). Preferably the device should power down very soon after active use and be able to power up again immediately upon continued use.

According to an embodiment of the invention, monitoring of the use status of the device is attained by providing means for sensing contact between the patient portion of the device or alternatively for sensing proximity of the patient portion of the device to a portion of the body of a patient. Thus, the underlying principle of the invention is monitoring of use status by contact or proximity detection.

The principles of some preferred embodiments of the invention are briefly summarised below:

(1) When the device-operator/patient portion is actually held in physical contact with a patient's body surface, the tremor (e.g. involuntary muscle tensions) either from the patient or from the user holding the device-operator/patient portion of the device will produce a low-frequency, high amplitude signal that stands out from the other signals typically picked up by the device-perator/patient portion (for instance the acoustic sensor of a stethoscope) and recorded or displayed by the device. In case of an electronic stethoscope, these low-frequency, high amplitude signals will clearly stand out from other sounds typically observed with the electronic stethoscope and when the stethoscope is not in use, the sound sensor will only be in contact with the surrounding air and only very little sound will be picked up, especially in combination with an ambient noise reduction transducer system, as described in international patent application WO2004/002191.

(2) When in combination with a sensor means comprising a piezoelectric transducer, such as the microphone component described in international patent application WO 2005/032212 A1, the capacitance of the piezoelectric device will change during physical deflection resulting from application of the sensor unto the body, and this change of capacitance may be detected and used for determining body contact between the patient portion and the sensor. Deflection of the piezoelectric element and hence the change of capacitance will vary with user and situation. Therefore in actual implementations, this principle may preferably be used for waking up the device followed by the acoustic check to precisely definer use status. It is important for the battery saving implementation that the wake-up may be performed without continuous use of DSP (digital signal processing) and capacitance change detection may be performed with substantially no current consumption.

(3) The invention may be based on capacitive proximity sensing that is able to detect a significant change in the (dielectric constant of the) medium connecting two individual electrodes of the proximity sensor, whereby the measured capacitance of the capacitor formed by the electrodes and intermediate medium will change measurably.

The use status of the device can according to the invention be monitored either continuously or periodically. Thus, the “intelligent” means required to read the physical sensor input (for instance a voltage/charge signal or capacitance from a piezoelectric sensor) may run continuously or only periodically in order to further minimise power consumption. For instance a periodic check of use status may be implemented using a high-power DSP waking up, for instance twice a second, running a fast check requiring in fact only a few milliseconds and then returning to stand-by/sleep mode. A continuous monitoring may require separate very low power electronic circuitry running continuously or whenever the high-power DSP is in stand-by/sleep mode.

The present invention may alternatively utilise at least the following detection principles to attain the monitoring and power consumption reducing objectives of the invention:

(4) Switch detection, for instance by monitoring the opening of the headset of an, electronic stethoscope, or activation of a switch when the patient portion is brought into contact with the appropriate portion of a patient.

(5) Strain gauge application sensing.

(6) Movement detection using for instance an accelerometer or gyroscope sensor means.

(7) Inductive detection, where detection is based on changes in magnetic properties, such changes being detected by for instance an integrated inductive coil in the stethoscope food of an electronic stethoscope.

(8) Monitoring of bio-impedance over the application surface.

(9) Proximity sensing using optical, ultrasonic or other sensor principles.

### BRIEF DESCRIPTION OF THE DRAWINGS

The present invention will be better understood with reference to the following detailed description of various embodiments of the Invention taken in conjunction with the figures, where:

FIG. 1 shows the stethoscope sensor (chestpiece) of an electronic stethoscope being held in contact with a surface portion of a patient;

FIGS. 2(a), (b) and (c) show plots of a sensor signal as a function of time, (a) showing the raw output signal provided by the sensor, (b) showing a low pass filtered version of this signal and (c) showing the RMS value of the output signal from the low pass filter with a sufficiently slow time constant used for calculating the RMS value;

FIGS. 3(a), (b) and (c) show the suppression of noise spikes by the low pass filter, (a) the raw signal, (b) the low pass filtered signal, and (c) the calculated RMS value of the signal.

FIGS. 4(a), (b) and (c) show another example of suppression of noise spikes by low pass filtration;

FIG. 5 shows a variable threshold value included in order to determine use status by comparison of the signal amplitude (RMS low pass filtered output signal from the sensor) with the threshold value;

FIG. 6 shows a plot illustrating acoustic detection of frictional noise in a signal provided by the sensor of FIG. 1, the plot showing the power spectral density as a function of frequency when frictional noise is present and when frictional noise is not present;

FIGS. 7(a), (b) and (c) show proximity sensing using capacitance means;

FIGS. 8(a) and (b) show bioimpedance sensing;

FIGS. 9(a) and (b) show a further example of use of the principles of the present invention relating to the application of a syringe;

FIG. 10 shows a further example of use of the principles of the present invention relating to the application of a syringe, and

FIG. 11 shows a further example of use of the principles of the present invention relating to the application of an inhaler device.

### DETAILED DESCRIPTION OF THE INVENTION

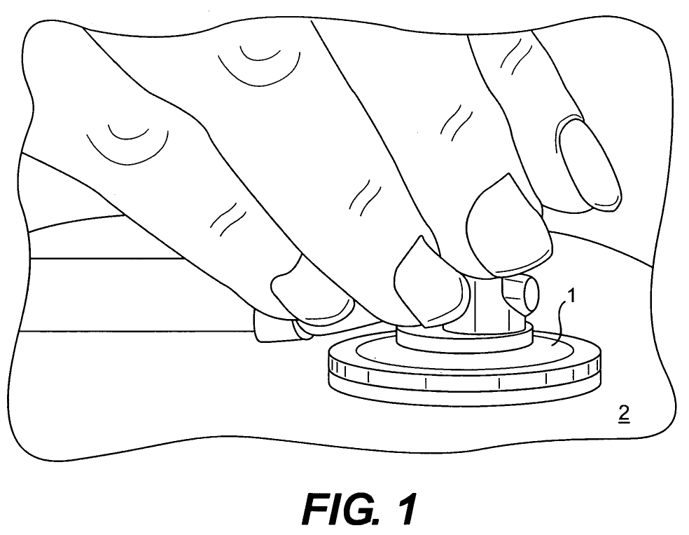

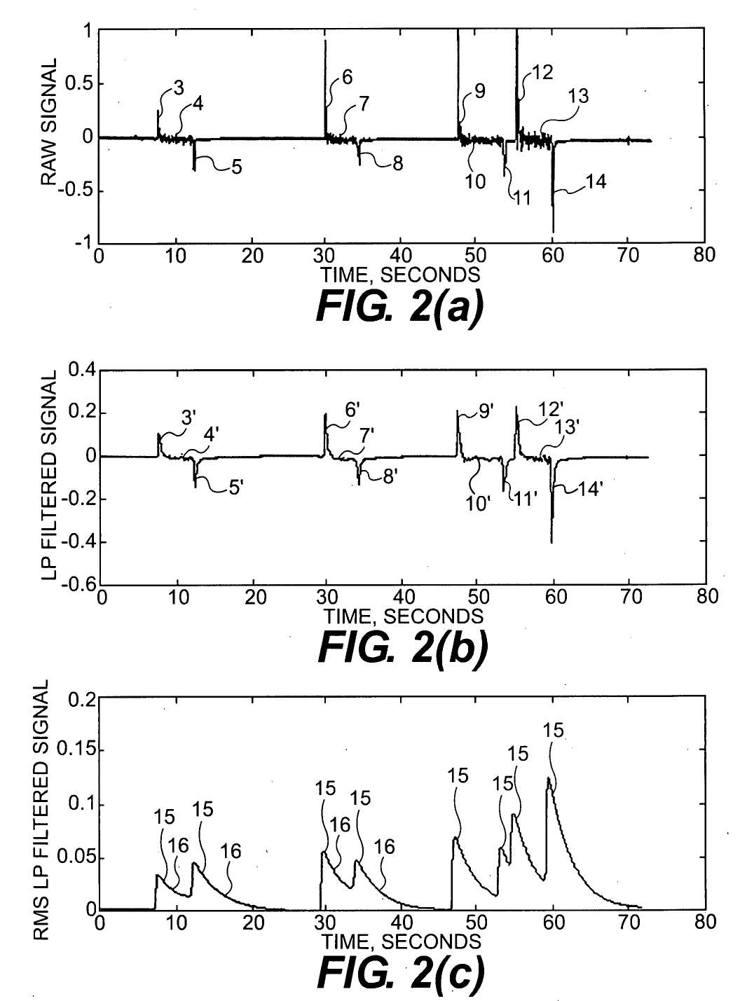

Referring to FIG. 1 there is shown the stethoscope sensor portion 1 (chestpiece) of an electronic stethoscope being held in contact with a surface portion 2 of a patient. When the stethoscope sensor is initially being brought into contact with a surface portion of a patient, the output signal provided by the sensor will initially exhibit a powerful peak when the sensor hits the surface portion of the patient. Subsequently, when the sensor is in contact with the surface portion, tremor originating from the patient or the user holding the sensor portion 1 will produce a low-frequency signal, that will stand out distinctly from other signals typically observed with the stethoscope. After use of the stethoscope, the sensor portion is again removed from the surface of the patient and this removal will result in a final powerful peak output signal being provided by the sensor. According to a first embodiment of the invention, the above described sequence of output signals from the sensor is used to monitor use status of the stethoscope, as will be described in more detail in connection with FIGS. 2 through 6.

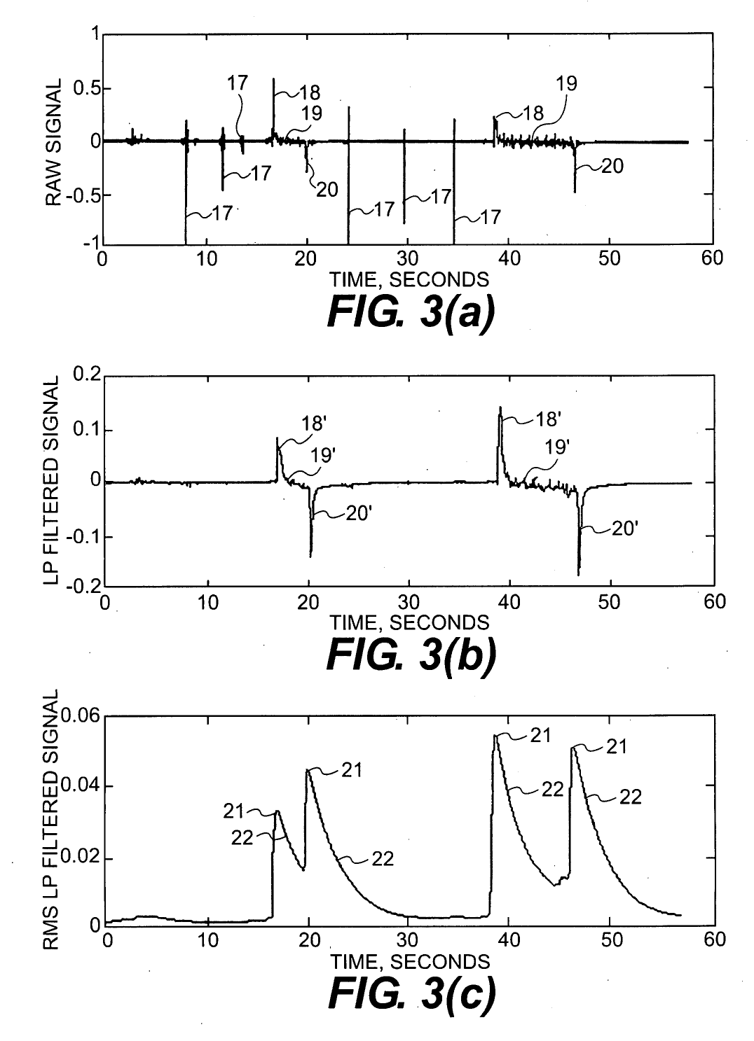

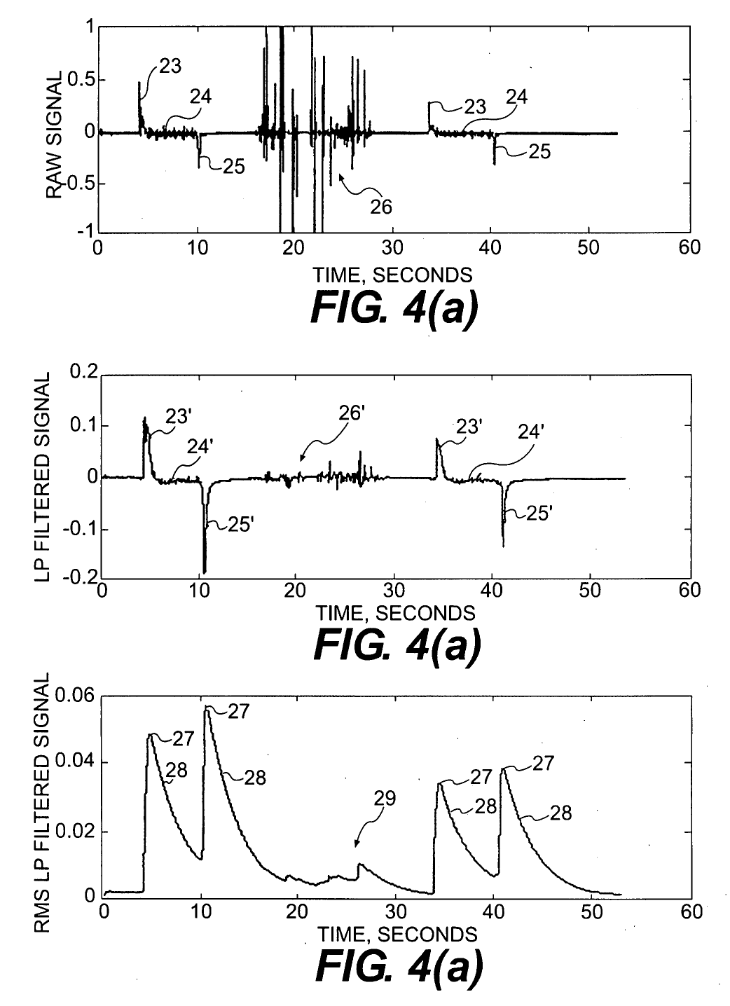

Referring to FIG. 2(a) there is thus shown the unprocessed output signal (shown in arbitrary units) from the stethoscope sensor 2 as a function of time during an exemplary use sequence. Initially the sensor is applied to the surface of a patient, giving rise to the short and relatively powerful peak 3 in the output signal from the sensor. The sensor is now in contact with the surface and provides an output signal 4 generated by vibrations (tremor) from the body of the patient or from the user holding the stethoscope or by body sounds (e.g. heart and lung sounds) of the patient. Finally, the sensor is removed from contact with the surface giving rise to the peak output at 5. This sequence of events is subsequently repeated as indicated by reference numerals 6, 7, 8; 9, 10, 11 and 12, 13, 14, respectively. In FIG. 2(b) high frequency components in the output signal from the sensor, for instance caused by noise not related to the vibration/tremor induced signal portions described above, have been removed by low pass filtration of the output signal from the sensor, maintaining the above pattern of initial contact pulses 3′, 6′, 9′ and 12′ followed by the vibration/tremor intervals 4′, 7′, 10′ and 13′ and the final contact-release pulses 5′, 8′, 11′ and 14′, respectively. Thus, the required information relating to the use status of the stethoscope is maintained after low pass filtration by choosing the type and characteristics of the low pass filter properly, but without the interfering high-frequency noise. The removal of severe noise components will be illustrated in connection with FIGS. 3 and 4 below, The low pass filter actually used in the shown example is a 1 Hz Butterworth LP filter, but other filter types or characteristics, such as cut-off frequencies could also have been used for instance according to the frequency content of the unwanted noise.

Referring to FIG. 2(c) there is shown a processed version of the signal in FIG. 2(b), where the RMS value of the signal in FIG. 2(b) has been calculated with a suitable time constant, thus providing a processed output signal comprising peaks 15 indicating establishing and release of contact between the sensor in the stethoscope and a surface portion of a patient. The time constant determines the sloping portions 16 of the processed signal shown in FIG. 2(c).

Referring to FIGS. 3(a), (b) and (c), a situation is illustrated, where powerful noise peaks occur in the output signal from the stethoscope sensor apart from the peaks caused by establishment and release of contact between the stethoscope sensor and a surface portion of a patient. Such extraneous noise peaks are in FIG. 3(a) shown at reference numeral 17 and these peaks occur randomly distributed along the time axis. Contact establishment followed by vibration/tremor periods and terminated by contact release is again indicated by reference numerals 18, 19 and 20, respectively. In FIG. 3(b) a low pass filtered version of the output signal shown in FIG. 3(a) is shown, from which it appears that the extraneous noise peaks 17 in the un-processed signal have been effectively removed leaving the required use-related sequences 18′, 19′, 20′ intact in the filtered signal. FIG. 3(c) shows calculated RMS value of the signal shown in FIG. 3(b) comprising peaks 21 indicating establishing and release of contact between the sensor in the stethoscope and a surface portion of a patient.

Referring to FIGS. 4(a), (b) and (c), a situation substantially corresponding to that of FIGS. 3(a), (b) and (c) is shown, but comprising an interval 26 containing very powerful peak noise as well as noise of a more steady-state nature It is important that this interval be not misinterpreted as an Interval of actual use of the stethoscope and hence that the low pass filter should be able to substantially suppress the noise signal in this interval. The noise suppression attained by low pass filtering is illustrated in FIG. 4(b), where only a weak residual noise signal 26′ is left. The resultant signal after RMS calculation is shown in FIG. 4(c) and the noise-infected portion 26 in the original, unprocessed signal in FIG. 4(a) has been substantially suppressed as indicated by reference numeral 29 to an extent that this interval will not be misinterpreted as a use sequence, actual use sequences being indicated by the signal portions 27 and 28 in FIG. 4(c), which portions clearly stand out relative to the signal in the interval 29.

When evaluating the numerical value of the use status determining signal, for Instance the RMS processed low pass filtered signal shown in FIGS. 2(c), 3(c) and 4(c) to determine the use status of the electronic stethoscope according to the invention, a threshold T (which can be varied/optimised according to specific requirements) is applied.

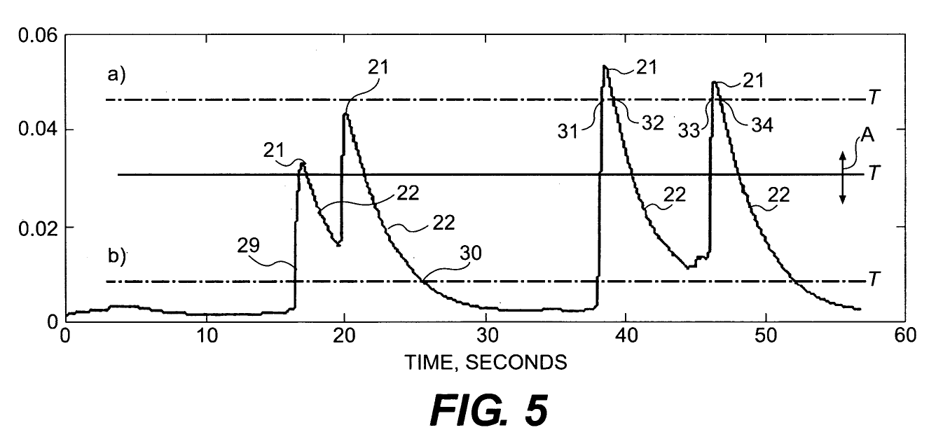

Referring to FIG. 5 there is shown the RMS low pass filtered signal also shown in FIG. 3(c) together with a variable threshold T that can be adjusted between a very high threshold value (a) and a very low threshold value (b). The threshold value (a) is so high that only the most powerful peaks of the signal will activate the stethoscope, whereas the threshold value (b) is so low that even very weak signals will activate the stethoscope. The achieved activation (signal above threshold) of the stethoscope may in some embodiments of the invention be combined with a timer circuit, whereby the stethoscope, once activated, will remain active for a given (user definable) period of time, for instance three minutes. Furthermore, this timed activation may only require a positive triggering for instance once every third minute for the stethoscope to remain active. Furthermore, different system strategies may enforce different ways of structuring the described timed activation: For instance the same type of system activation could be achieved either by combining a high threshold value (a) with a relatively long time-out period, or by combining a low threshold value (b) with a significantly shorter time-out period, as a type (b) threshold setting would be more likely to be exceeded by the signal than a type (a) threshold setting.

When the means and principles of the Invention are used in an electronic stethoscope as a means to identify when the stethoscope is being applied on a patient's chest and hence brought into the active use state, it is critical that there is provided a very robust detection of activity, e.g. threshold type (b), so that the stethoscope always activates promptly. Once activated, it is reasonable that the system follows the standard time-out power-down period of time, for instance three minutes.

In stethoscope applications where the battery lifetime is very critical this three minute period may be unacceptable, and hence additional rules could be employed. Thus it could for instance be required that the period in which the signal (for instance the RMS low pass filtered amplitude of the signal) exceeds the type (b) threshold value is longer than a given time value, for instance two seconds, before the said three minutes period activation is enabled. Alternatively, it could for instance be required that a signal in excess of the type (a) threshold value must occur twice within a given period of time, for instance two seconds, before a three minutes system activation is enabled.

A still further system activation strategy would be to always let the system be as easy to activate as possible, i.e. using a simple type (b) threshold value activation, and additionally use a sufficiently detailed analysis of the characteristics of the signal (frequency spectrum, details of temporal structure, etc.) to determine whether the detected signal with a high probability could be caused by the stethoscope sensor being in fact in contact with a patient's chest. This more advanced analysis could for instance comprise detection of the patient's heartbeat or detection of respiratory sounds, etc., which sounds must occur within a predetermined period of time, for instance a few seconds, for the stethoscope to remain in the active state. If such sounds do not occur within the said interval, the stethoscope will power down in order to preserve battery lifetime.

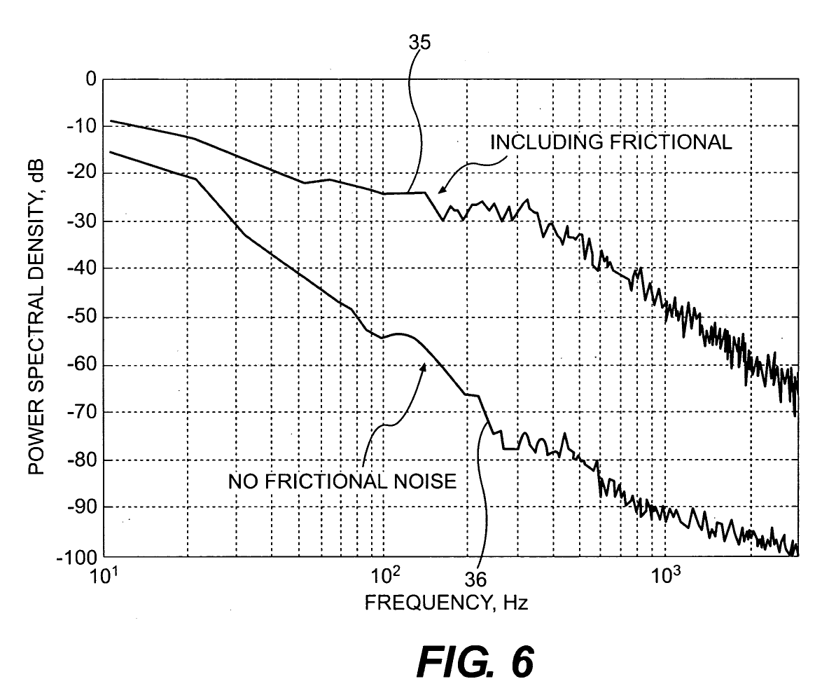

In the embodiment of the invention described in detail above, determination of use status was based on the signal components that typically occur when the stethoscope sensor is being brought into contact with a surface portion of a patient (indicated by the initial output signal peak, for instance 3 in FIG. 2(a)), remains in contact with this surface portion (for instance the vibration/tremor-induced signal portion 4 in FIG. 2(a)) and at removal of the sensor from contact with this surface portion (indicated by the final output signal peak, for instance 6 in FIG. 2(a)). Alternatively or supplementary to this use status determination method, sound components in the output signal from the stethoscope (or other device as mentioned in the following) originating from friction for instance between the sensor of the stethoscope and the surface portion of the patient could be used for determining use status of the stethoscope or other device. An example of such frictional noise is shown in FIG. 6, where the power spectral density (dB) is shown as a function of frequency for an output signal from a stethoscope sensor when frictional noise is present in the output signal (reference numeral 35) and when frictional noise is not present in the output signal (reference numeral 36). Referring to FIG. 6 it clearly appears that frictional noise contains more or more powerful high-frequency components than normal auscultation sound that will be picked up by the stethoscope sensor when no friction for instance between the sensor and the surface portion of a patient occurs. Thus, for instance, sudden changes in the balance between the levels of a high-frequency portion of the power spectral density of the output signal from the sensor and a low frequency portion hereof could indicate a noise event and hence be utilised to provide information about the use status of the stethoscope or other device.

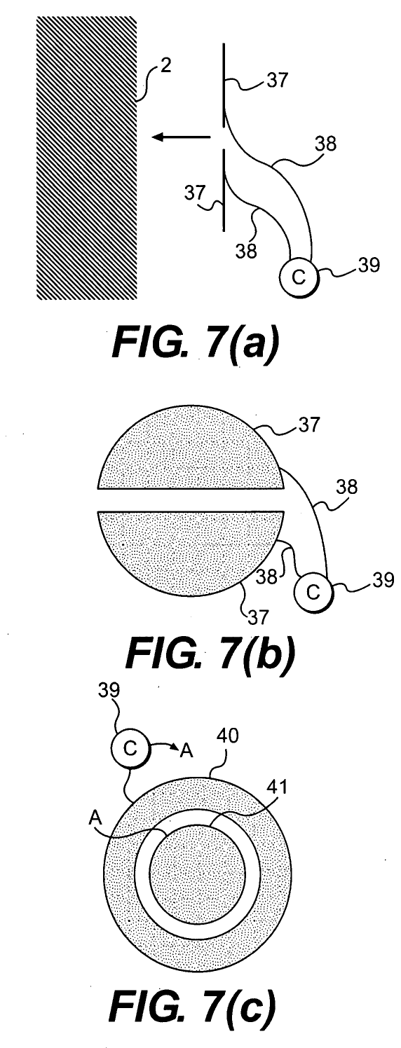

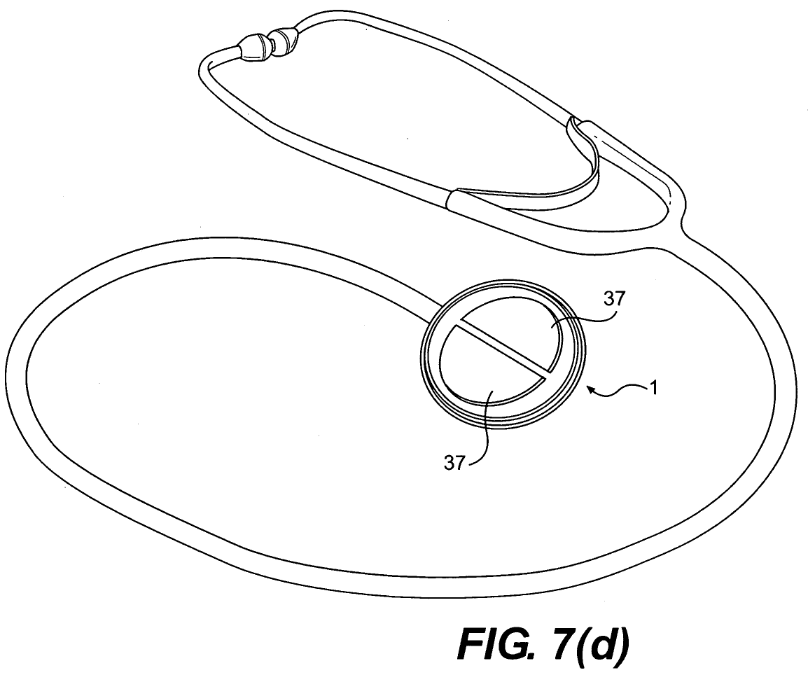

The above embodiments of use status determining means have basically relied on the pick-up of sound signals generated by vibration of the sensor or by physical impacts between the sensor and a surface portion of a patient. Referring now to FIG. 7(a) there is shown an alternative embodiment of use status determining means relying on proximity sensing using capacitance means. The capacitance between two electrodes 37 of a capacitor will change when the electrodes approaches a medium 2 (for instance human skin or tissue) with dielectric properties differing from air. Thus, providing the sensor portion of for instance an electronic stethoscope with an arrangement of electrodes, the capacitance changes when the sensor approaches a surface portion of a patient can be used to determine use status of the stethoscope. It might be advantageous to use adaptive threshold sensing for better handling of different use patterns, for instance pressing the sensor of the stethoscope harder or lighter against the surface portion of a patient. It is important that activation of the stethoscope is brought about by the sensor actually approaching the surface portion of the patient and that activation is not brought about by proximity of the sensor of the stethoscope (or other parts of a stethoscope or other medical devices) to the operator himself, by for instance sensing the hand of the operator closing around the device. As shown in FIG. 7(b), the electrodes (37 in FIG. 7(b) and 40 and 41 in FIG. 7(c)) used for implementing a proximity sensor can be arranged in different manners, of which only two are shown, according to the specific requirements of the device. The electrodes are via electrical connectors 38 connected to impedance sensing means 39.

An embodiment of a stethoscope comprising said capacitance based proximity detecting means is shown in FIG. 7(d). In this embodiment, two capacitor electrodes 37 are positioned in the sensor portion 1 of the stethoscope as close as possible to the external media in order to optimally utilise the change in the external medium's dielectric properties to change the capacitance formed by the electrodes. The electrodes need not be in galvanic connection with the external medium but may be hidden behind a moisture protection diaphragm (not shown). The electrodes may be applied to the surface of a patient interface polymer diaphragm by means of thin metal/conductive layers, for instance obtained through a silk-print application process. Internal electronic circuitry in the sensor portion or otherwise provided in the stethoscope is provided for detecting the resultant capacitance and/or changes hereof and for utilising such capacitance or changes for determining use status of the stethoscope

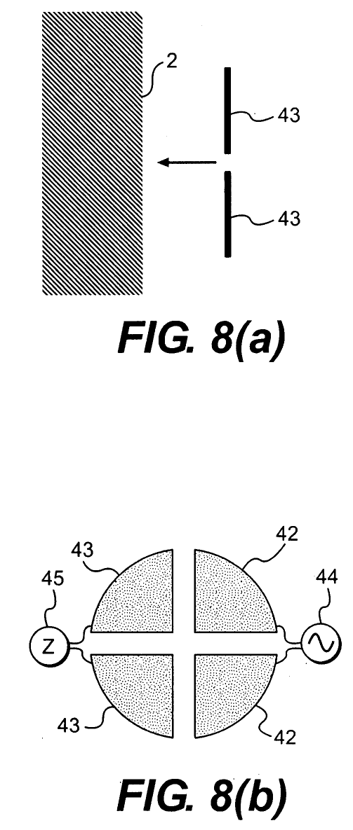

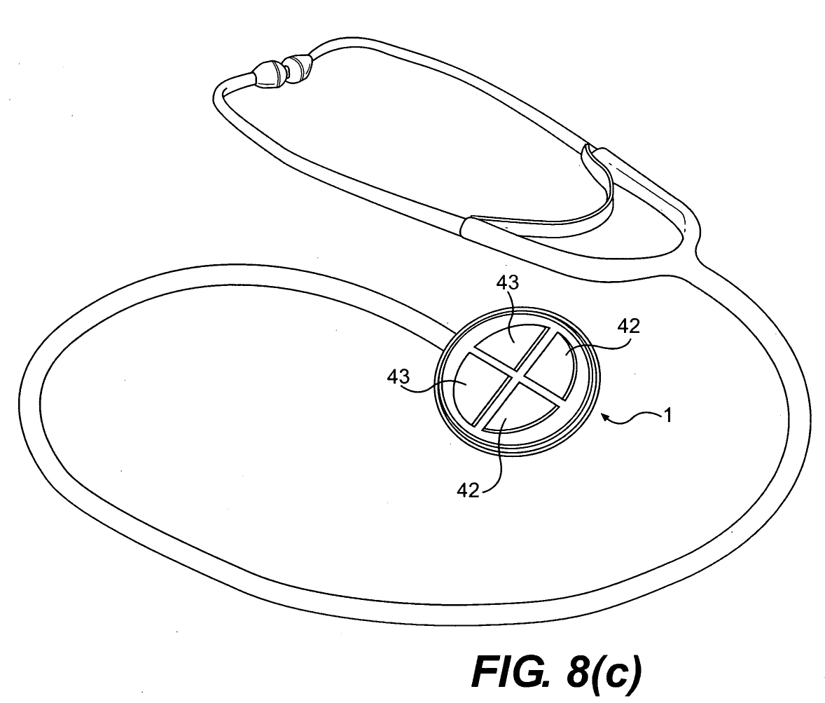

Referring to FIGS. 8(a) and (b) there is shown the application of bioimpedance sensing for determining use status of an electronic medical device, such as a stethoscope. In order to carry out four-pole impedance measurements, two electrodes 42 couple electrical energy to the patient's tissue at constant electrical current provided by a signal (current) source 44. Two other electrodes 43 are used for measuring voltage drop over a chosen tissue area. The shown bioimpedanse-sensing device requires electrical contact between the various electrodes and the application site on/in a patient. The said voltage drop can be measured by means 45 connected to the pair of electrodes 43. It is understood that the shown configuration of electrodes and the actual shape of these electrodes are only exemplifying and that numerous alternative shapes and configurations of electrodes could be used. Typically the signal applied from the source 44 will be a periodic signal, for instance sinusoidal, of 50 kHz in order to provide a good estimate of conductivity through human (water) fluid.

Use status determination based on the application of bioimpedance sensor means can for instance be used in connection with inhaler devices, where the sensor means can be used for sensing proper closing of the user's lips around the inhaler mouthpiece. Bioimpedance-sensor means can also be used for sensing proper insertion of an injection pen into human (or other) tissue or for sensing a hand of an operator touching the medical device and turning on the device accordingly.

The application of bioimpedance sensing specifically for determining use status of an electronic stethoscope is shown in FIG. 8(c). The sensor portion 1 of an electronic stethoscope is provided with the four-pole-impedance measurement electrodes 42 and 43 in such a manner that galvanic contact with the skin of the patient will be provided when the stethoscope is used. The electrodes may be applied to the surface of a patient interface polymer diaphragm by means of thin metal/conductive layers, e.g. obtained through a silk-print application process. Internal electronic circuitry provides means for detecting the resulting bioimpe dance using an optimised setting of the stimulus signal 44, e.g. with regard to frequency and/or amplitude. Typically an AC stimulus signal frequency of approximately 50 kHz will allow for optimised low current requirements, resulting in a safe system.

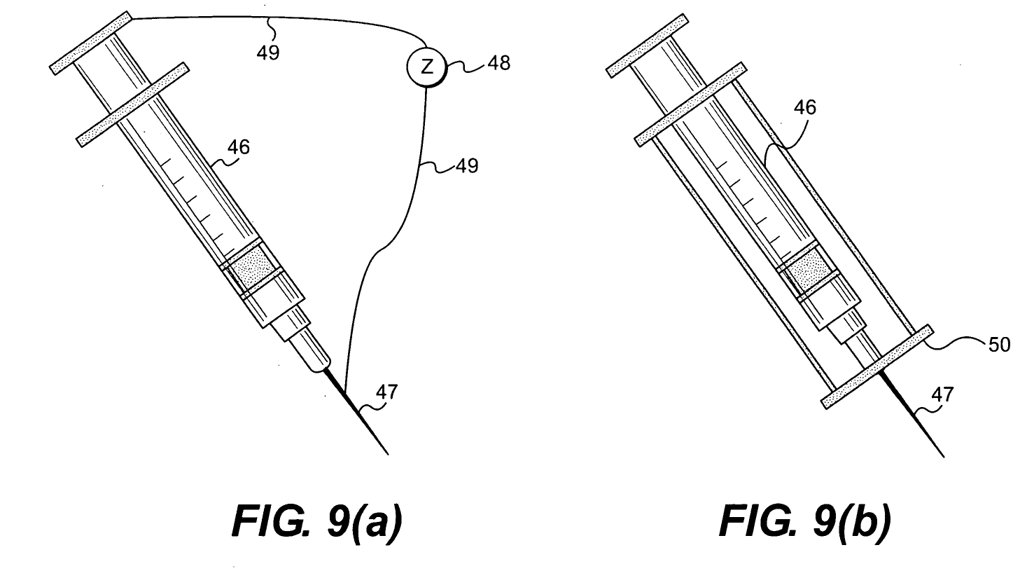

Referring to FIGS. 9(a), 9(b) and 9(c) there is shown a further example of use of the various functional principles (two-pole impedance measurement, vibration sensing, capacitance proximity sensing and four-pole bioimpedance sensing) described above for determining the use status of an auto-injection pen device, i.e. for ensuring proper needle insertion into the tissue of a patient before firing the device. Thus, specifically FIG. 9(a) illustrates the use of two-pole impedance measurement between the main body 46 of an auto-injection pen device and the needle portion 47 hereof. When the needle 47 is inserted in the patient's tissue, an electrically conductive pathway is established through the patient's body, under the assumption that it is the patient himself that actually operates the device. Prior to use, when the needle 47 is not in contact with the tissue of the patient, a very high—substantially infinite—impedance between the needle 47 and the main body 46 of the device can be determined by impedance-measuring means 48 provided within the housing of the device, and when contact between the needle 47 and the tissue of the patient Is established, this impedance drops substantially. This drop in impedance is according to the invention utilised for providing the required information about use status of the device. Alternatively, vibration of the needle 47 relative to the main body 46 or vibration of an interface plate 50 of the device may be picked up by vibration-sensitive means substantially in the same manner as described in connection with the stethoscope application Illustrated in FIGS. 2 through 5.

Alternatively, the interface plate 50 may be provided with capacitance proximity-sensing means, substantially as described in connection with a stethoscope in FIG. 7 above, for sensing proximity of the interface plate 50 to the surface of a patient. Also the bioimpedance-measuring means described above in connection with FIG. 8 could be incorporated into the interface plate 50 to determine when contact is made between the interface plate of the device and the skin surface of a patient.

By the above means, the use status: “device ready for firing” can be determined. It is furthermore possible to use the above means to ensure that the needle is kept in the tissue of the patient for a required period of time prior to retraction of the needle. The simple two-pole Impedance measurement of FIG. 9(a) is a straightforward manner of accomplishing this aim.

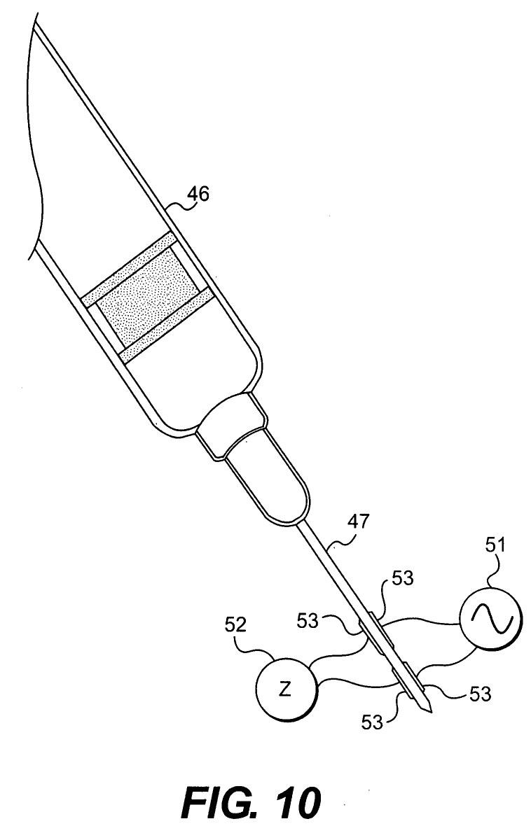

Referring to FIG. 10 a further application of bioimpedance-sensing means in connection with an auto-injection pen device is illustrated. Based on a more detailed analysis of tissue impedance in muscle, fat, arteries, veins, etc. the correct positioning of the needle in the tissue of a patient can be monitored by means of a four-pole bioimpedance analysis. Thus, for instance the electrical impedance of fat is much higher than the impedance of muscle tissue or fluids flowing in arteries and veins. As shown in FIG. 10, four individual electrodes 53 are positioned in the vicinity of the tip of the needle 47 by means of a suitable surface-mount technique, including the steps of first covering the entire needle 47 with an electrically insulating layer and then applying (for instance by silk-print or by a suitable photographic process) the four individual electrodes 53 provided with contact interfaces at the top portion of the needle. Finally, the entire area of the electrodes is covered with an electrically insulating layer only leaving the outer surface portions of the electrodes open for contact with the surrounding tissue. Two of the electrodes 53 are as previously used to provide an excitation signal 51 to the electrodes and the impedance of the tissue portion in contact with the electrodes is measured by suitable means 52 provided in the auto-injection pen device.

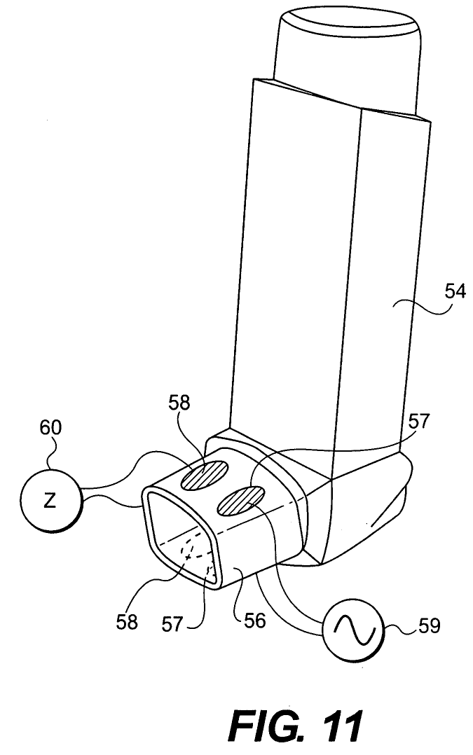

Referring to FIG. 11 there is illustrated a further example of use of the principles of the present invention for monitoring use status of an inhaler device 54. The inhaler device comprises the main body 54 and the mouthpiece 56 and by using the use status sensing means described in previous paragraphs of this specification, proper folding of the lips of a user around the inhaler mouthpiece 56 prior to the release of a dose of medicament from the inhaler device can be ascertained. Thus, medication is prevented from being delivered to the surrounding air through leakages between the lips of the user and the mouthpiece of the device. As shown in FIG. 11, four-pole sensing of the impedance of the appropriate portions of a user's lips when in contact with the surface of the mouthpiece is carried out by means of pairs of electrodes 57, 58, one electrode of a pair provided on the upper surface of the mouthpiece as seen in FIG. 11 and the other opposite the first, i.e. on the bottom surface of the mouthpiece. Two of the electrodes, 57, serve to provide the excitation signal 59 to the bioimpedance-measuring means and the other two electrodes 58 are used for measuring the bioimpedance through the lip portion of the user. Alternatively, two electrodes could have been used to carry out two-pole impedance measurements of the lip portion of the patient to monitor correct contact between the lip portion of the user and the mouthpiece. As a further alternative, vibration sensing of the mouthpiece, as described in connection with the previous stethoscope example in FIGS. 2 through 5, could be used for determining if the mouthpiece is in physical contact with any external objects, such as the users lip portions.

Supplementary or alternatively to using the bioimpedance or vibration sensor means in the manner described above, such means could according to the invention be used to sense the user's hand, when the user holds the (main body of) the device. The provision of information of this use status, i.e. the user is actually holding the device, could be used for turning on backlight on a LCD display and/or initiate text guidance on the display relating for instance to proper inhalation technique, time since last dose from the inhaler, etc. For this purpose, two-pole or four-pole bioimpedance sensing using electrodes appropriately placed on the inhaler main body, i.e. in those regions of the main body where the user's hands/fingers touch the main body), could be used. Alternatively, vibration sensors in the housing of the inhaler device could be used for detecting the faint muscle tremor occurring from the user holding the device by hand.

1. A method for automatically determining use status of an electronic medical device and/or activating said electronic medical device, such as an electronic stethoscope, the method comprising providing a patient portion of the device, i.e. one or more portions of the device that during use is brought into contact with portions of a patient, with contact or proximity detector means that provides an output signal when said patient portion is proximate to or in contact with a portion of a patient, said output signal, optionally after a predetermined signal processing, providing a signal-processed version of said output signal, determining the use status of the device.

2. A method according to claim 1, where said output signal or said signal-processed version hereof is used for activating electronic signal-processing circuitry, such as amplifiers, filters, signal analysis means, etc., whereby said electronic medical device becomes active when said patient portion is brought into contact with a portion of a patient or brought into close proximity of a patient.

3. A method according to claim 2, where said signal processing of said output signal comprises low pass filtration of said output signal, whereby a low pass filtered version of said output signal is provided.

4. A method according to claim 3, where said low pass filtered version of said output signal is processed by RMS (root mean square) determining means with a suitable time constant, whereby a RMS value of said low pass filtered version of said output signal is provided.

5. A method according to claim 2, where said signal processing of said output signal comprises evaluation of the balance between the levels of a high-frequency portion of the power spectral density of said output signal and a low frequency portion of the power spectral density of said output signal, whereby the presence of frictional noise components in said output signal can be evaluated.

6. A method according to claim 1, where said detector means is a vibration sensor.

7. A method according to claim 1, where said vibration sensor is a piezoelectric sensor.

8. A method according to claim 7, where said piezoelectric sensor provides an output signal that is amplified by a low-power amplifier, such as a FET, MOSFET, bipolar operational amplifier.

9. A method according to claim 1, where said detector means is a capacitance proximity sensor.

10. A method according to claim 1, where said detector means is a bio-impedance sensor.

11. A method according to claim 10, where said bio-impedance sensor is a two-pole sensor.

12. A method according to claim 10, where said bio-impedance sensor is a four-pole sensor.

13. A method according to claim 1, where said medical device is an electronic stethoscope.

14. A method according to claim 1, where said medical device is an electronic auto-injector device.

15. A method according to claim 1, where said medical device is an electronic inhaler device.

16. An electronic stethoscope comprising a chestpiece comprising a stethoscope sensor for picking up sounds from a patient's body, where said chestpiece is provided with contact or proximity detector means that provides an output signal when said chestpiece is proximate to or in contact with a surface portion of a patient, said output signal, or a signal-processed version of said output signal, determining the use status of the device and/or activating the stethoscope when said chestpiece is proximate to or in contact with said surface portion of a patient.

17. An electronic stethoscope according to claim 16, furthermore comprising amplification means and/or other electronic signal-processing means for amplifying/processing output signals from said stethoscope sensor, where said amplification means/processing means are turned on when said contact or proximity detector means determines that said chestpiece is in contact with said surface portion of a patient or when said chestpiece is in proximity to said surface portion of a patient.

18. An electronic stethoscope according to claim 16, where said contact detector means is a vibration sensor, where the vibration sensor will generate a voltage or charge upon physical contact with the body of the patient, which voltage/charge can be used to trigger status setting of the electronic stethoscope.

19. An electronic stethoscope according to claim 18, where said vibration sensor is a piezoelectric vibration sensor.

20. An electronic stethoscope according to claim 19, where said piezoelectric vibration sensor is in combination with a low-power amplifier means, such as a FET, MOSFET, bipolar operational amplifier, where the piezoelectric sensor will generate a voltage/charge upon physical contact with the skin of the patient, which voltage/charge is amplified by said low-power amplifier means.

21. An electronic stethoscope according to claim 16, where said detector means is a capacitance proximity sensor, where the capacitance increases when the sensor approaches the body of a patient.

22. An electronic stethoscope according to claim 16, where said use status of the stethoscope is determined by means capable of determining the bio-impedance at an interface area between the chestpiece of the stethoscope and a surface portion of a patient, where said bio-impedance is reduced when the patient chestpiece of the stethoscope touches said surface portion of the patient.

23. An electronic stethoscope according to claim 22, where said bio-impedance is determined by two-pole impedance-determining means.

24. An electronic stethoscope according to claim 22, where said bio-impedance is determined by four-pole impedance-determining means.

25. An electronic stethoscope according to claim 16, where said output signal that indicates when said chestpiece is proximate to or in contact with a surface portion of a patient is provided by the stethoscope sensor itself.

26. An electronic stethoscope according to claim 16, where said signal processing comprises low pass filtration of said output signal, whereby a low pass filtered version of said output signal is provided.

27. An electronic stethoscope according to claim 26, where said low pass filtered version of said output signal is processed by RMS (root mean square) determining means with a suitable time constant, whereby a RMS value of said low pass filtered version of said output signal is provided.

28. An electronic stethoscope according to claim 16, where said signal processing of said output signal comprises evaluation of the balance between the levels of a high-frequency portion or band of said output signal and a low frequency portion or band of said output signal, whereby the presence of frictional noise components in said output signal can be evaluated.

29. An electronic stethoscope according to claim 16, where said output signal, or a signal-processed version of said output signal, determines the use status of the device and/or activates the device when said output signal or processed version hereof exceeds a given threshold value.

30. An electronic stethoscope according to claim 29, where said threshold value is variable.

31. An electronic stethoscope according to claim 16, where the stethoscope after activation is automatically turned off after a given period of time.

32. An electronic auto-injection device comprising a main body and a needle, where the device is provided with detection means for detecting an electronically conductive pathway established between said needle and said main body of the device when the needle is inserted into the tissue of a patient, said detecting means providing an output signal, where the output signal, or a signal-processed version hereof, determines the use status, such as proper insertion of the needle into a patient's tissue, of the auto-injection device.

33. An electronic auto-injection device according to claim 32, where said detection means comprises two-pole or four-pole impedance-sensing means.

34. An electronic auto-injection device comprising a main body and a needle, where the device is provided with vibration sensing means for sensing the vibration of the needle and/or the vibration of the device main body, said vibration sensing means providing an output signal, where the output signal, or a processed version hereof, determines the use status of the device.

35. An electronic auto-injection device comprising a main body, a needle and an interface plate for providing an interface between the device and a surface portion of a patient, where said interface plate is provided with capacitance proximity sensing means for sensing proximity of the interface plate to a surface portion of a patient, where the capacitance proximity sensing means provides an output signal, where the output signal, or a signal-processed version hereof, determines the use status of the device.

36. An electronic auto-injection device according to claim 35, where said output signal, or said signal-processed version hereof, determines the time interval between insertion and retraction of the needle, whereby it can be monitored whether the needle has been kept in the tissue of a patient for a required period of time.

37. An electronic auto-injection device according to claim 35, where the device is furthermore provided with signal analysis means for discrimination between needle insertion into a patient's muscles or fat or into an artery or vein, where said analysis means receives the measured impedance means from said detection means or two- or four-pole impedance-sensing means and based on these received impedance measurements differentiates between needle insertion in muscle, fat, arteries or veins.

38. An electronic inhaler device comprising a mouthpiece, where the mouthpiece is provided with two- or four-pole impedance-measuring means formed for contact with the mouth or lip portion of a patient, wherein the measured impedance provides information about the use status, such as whether the lips of the patient are properly folded around the mouthpiece, of the inhaler device.

39. An electronic inhaler device comprising a mouthpiece, where the device is provided with vibration-sensing means for providing an output signal when the mouthpiece of the device is subjected to vibrations caused by contact of the mouthpiece with lip portions of a patient, where the vibration-sensing means provides an output signal indicating such vibration, and where the output signal, or a signal-processed version hereof, indicates use status of the device.

40. An electronic inhaler device comprising a mouthpiece and a hand grip portion, where the hand grip portion is provided with either two- or four-pole impedance-sensing means or with vibration-sensing means, said means providing an output signal indication when a person is holding said hand grip portion, where said output signal, or a signal-processed version hereof, is used for turning on an LCD display and/or for initiating a text guidance on the display relating for instance to proper inhalation technique and/or time elapsed since last dose of medicament provided by the device.

41. An electronic auto-injection device according to claim 32, where said output signal, or said signal-processed version hereof, determines the time interval between insertion and retraction of the needle, whereby it can be monitored whether the needle has been kept in the tissue of a patient for a required period of time.

42. An electronic auto-injection device according to claim 32, where the device is furthermore provided with signal analysis means for discrimination between needle insertion into a patient's muscles or fat or into an artery or vein, where said analysis means receives the measured impedance means from said detection means or two- or four-pole impedance-sensing means and based on these received impedance measurements differentiates between needle insertion in muscle, fat, arteries or veins.

43. An electronic auto-injection device according to claim 34, where said output signal, or said signal-processed version hereof, determines the time interval between insertion and retraction of the needle, whereby it can be monitored whether the needle has been kept in the tissue of a patient for a required period of time.

44. An electronic auto-injection device according to claim 34, where the device is furthermore provided with signal analysis means for discrimination between needle insertion into a patient's muscles or fat or into an artery or vein, where said analysis means receives the measured impedance means from said detection means or two- or four-pole impedance-sensing means and based on these received impedance measurements differentiates between needle insertion in muscle, fat, arteries or veins.
